(19) United States
(12) Patent Application Publication
(10) Pub. No.: US 2020/0306292 A1
(43) Pub. Date: Oct. 1, 2020

(54) METHOD FOR TREATING ACUTE HYPOVOLEMIA

(71) Applicant: Wonder Spray, LLC, San Diego, CA (US)

(72) Inventor: John Burd, San Diego, CA (US)

(73) Assignee: Wonder Spray, LLC, San Diego, CA (US)

(21) Appl. No.: 16/508,094

(22) Filed: Jul. 10, 2019

Related U.S. Application Data
(62) Division of application No. 16/363,756, filed on Mar. 25, 2019.

(51) Int. Cl. A61K 33/20; A61K 9/00; A61M 1/16; A61M 1/28

(52) U.S. Cl. CPC A61K 33/20; A61M 1/287; A61M 1/1654; A61K 9/0019

(57) ABSTRACT

A method of treating acute hypovolemia due to one or more medical conditions including sepsis with shock, hemorrhagic shock, burn injury, nephritic syndrome, and multiorgan failure by injecting an aqueous solution of hypochlorous acid to a patient in need thereof.

### CROSS-REFERENCE TO RELATED APPLICATIONS

The present application is a divisional of U.S. patent application Ser. No. 16/363,756, filed on Mar. 25, 2019, the content of which is incorporated herein by reference in its entirety.

### TECHNICAL FIELD

The present disclosure is generally related to suppressing pathogen activity and hypovolemia treatment, and more specifically, embodiments of the present disclosure relate to the use of hypochlorous acid (HOCl) in preparations of a pathogen inhibiting and resuscitation fluids as a treatment of sepsis, septicemia, and/or septic shock.

### BACKGROUND

Sepsis is a common and life-threatening inflammatory response to a severe infection anywhere in the body, such as pneumonia, influenza, or urinary tract infections. Both bacterial and viral infections can cause sepsis. Sepsis can be treated by an administration of antibiotics and fluid replacement.

Antiseptics agents are known to destroy or inhibit the growth and development of microorganisms in or on living tissue. Unlike antibiotics that act selectively on a specific target, antiseptics have multiple targets and a broader spectrum of activity, which include bacteria, fungi, viruses, yeast, mold, protozoa, spores, archaea, algae, and even prions. Several antiseptic categories exist, including alcohols (ethanol), anilides (triclocarban), biguanides (chlorhexidine), bisphenols (triclosan), chlorine compounds, iodine compounds, silver compounds, peroxygens, and quaternary ammonium compounds. The most commonly used products in clinical practice today include povidone iodine, chlorhexidine, alcohol, acetate, hydrogen peroxide, boric acid, silver nitrate, silver sulfadiazine, and sodium hypochlorite. Chlorine-based compounds have been traditionally used for both antiseptic and disinfectant purposes.

### SUMMARY

In accordance with one or more embodiments, various features and functionality can be provided to enable or otherwise facilitate preparations of HOCl used as a pathogen inhibiting and resuscitation fluid for treating sepsis, septicemia, and/or septic shock.

In some embodiments, a method for treating acute hypovolemia due to one or more medical conditions may comprise injecting an aqueous solution comprising a therapeutically effective amount of hypochlorous acid to a patient in need thereof.

In some embodiments, the method for treating acute hypovolemia may include injecting the therapeutically effective amount of hypochlorous acid within the aqueous solution comprising approximately 0.01% to 0.05% of the hypochlorous acid.

In some embodiments, the one or more medical conditions associated with hypovolemia may include sepsis with shock, hemorrhagic shock, burn injury, nephritic syndrome, and multiorgan failure, and/or other similar conditions. In some embodiments, the one or more medical conditions may be associated with third space fluid loss.

In some embodiments, the hypochlorous acid may have a pH range of approximately 6.1 to 6.3. In some embodiments, the hypochlorous acid is dissolved in a buffer solution. In some embodiments, the buffer solution may have buffering capacity for maintaining a pH of approximately 6.1 to 6.3.

In some embodiments, the buffer solution may include a phosphate buffer (e.g., a phosphate buffered saline), acetate buffer, citrate buffer, borate buffer, and combinations thereof.

### DETAILED DESCRIPTION

Sepsis is an extreme systemic inflammatory response to an infection. Sepsis can be caused by one or more infection-causing pathogens directly present in the bloodstream (septicemia) or as a response to an infection present only in one part of the body (e.g., pneumonia in lungs). This type of a systemic response to infection can affect virtually any organ system, including the central and peripheral nervous systems. In general, most chronic disease states can predispose to sepsis, e.g., diabetes, chronic liver or kidney disease, malignancy, or use of immunosuppressive medications.

Septic shock is a severe sepsis with acute circulatory failure characterized by persistent arterial hypotension (low blood pressure) despite adequate volume administration, unexplained by causes other than sepsis. As blood pressure falls, tissues become starved for oxygen-rich blood leading to organ failure and even death.

Septicemia was listed as the eleventh leading cause of death in the United States. More than 750 000 cases of sepsis are admitted every year to American hospitals, with more than half of these developing septic shock, ultimately resulting in some 258 000 deaths. Its incidence is thus comparable to the number of people developing the first heart attack (875 000) or stroke (700 000).

Despite advances in therapy and given an increasingly aging population that is especially susceptible to sepsis, the overall mortality rates remain relatively unchanged. The annual inpatient costs of treating septicemia were estimated at nearly $24 billion in 2013, an increase of $10 billion in 2008. Additionally, sepsis was responsible for 6.2 percent of all hospital costs, suggesting that the costs of patient care are only expected to grow.

Traditionally, sepsis is treated with antibiotics and fluid resuscitation. Given the high mortality rate, an early intervention response is critical. Once the systemic inflammatory response or sepsis manifesting in organ dysfunction, hypotension, and/or sepsis-induced tissue hypoperfusion has been recognized, establishing and maintaining adequate perfusion and giving early antibiotics is crucial.

Indeed, intravenous antibiotic therapy must be started as early as possible (e.g., within the first hour of recognition of septic shock and severe sepsis). Notably, antibiotic administration is often delayed. One of the reasons for the delay in administering antibiotic is failure to appreciate the risk of resistant organisms in certain scenarios. That is, failure to recognize that administration of inappropriate antimicrobials is essentially equivalent to absent antimicrobial therapy. Accordingly, choice of antimicrobials is crucial, as the administration of inappropriate therapy without activity for the given pathogen is equivalent to no therapy at all.

Recent review of sepsis cases demonstrated an increase in a rate of sepsis due to fungal organisms by 207 percent within the last 30 years, while gram-positive bacteria has been the predominant pathogen. Management of bacterial infection is especially critical for those with compromised immune system, for elderly and children, or for those suffering from physical trauma (e.g., severe burns) or long-term illnesses (e.g., diabetes, cancer, or liver disease) that may require treatment (e.g., hemodialysis), or for hose residing in a nursing home. Additionally, more severe septic infections occur in people who are already hospitalized with another illness or condition, or people who have a weak immune system. Pseudomonades are fairly common pathogens involved in infections acquired in a hospital setting.

Traditionally, fluid resuscitation has been considered an essential component of early sepsis management. Historically, patients with sepsis have received significant volumes of fluid throughout their ICU stay. Observational studies report positive fluid balances of five to eleven liters in the week after presentation. After resuscitation, potential benefits of fluid are balanced against risks of pulmonary edema, renal parenchymal edema, and effects of the IV fluid constituents themselves.

As alluded to above, a first-line treatment for patients with severe sepsis and/or septic shock includes timely administration of a pathogen suppressing agent along with fluid replacement therapy. Accordingly, an urgent need for a preparation that has both antimicrobial and/or antifungal properties and can also serve as a resuscitation fluid exists.

Embodiments of the technology disclosed herein are directed to fluid preparations comprising hypochlorous acid (HOCl) and delivery thereof as a treatment of sepsis, septicemia, and/or septic shock caused by microbial (including spores), viral, fungal, allergy-causing agents while increasing intravascular volume.

HOCl is a weak acid that forms when chlorine dissolves in water, and itself partially dissociates, forming hypochlorite, OCl−. Similar to other chlorine-releasing agents (e.g., sodium hypochlorite, chlorine dioxide, and the N-chloro compounds such as sodium dichloroisocyanurate), aqueous chlorine solution is well known for its antimicrobial, anti-inflammatory, and immunomodulatory properties.

Applications of aqueous solutions containing approximately 30-2500 ppm (0.003% to 0.25%) HOCl have been used in a variety of areas including (but not limited to) wound care, as antimicrobial agents.

HOCl is a potent antimicrobial capable of eradicating bacteria including antibiotic-resistant strains, viruses, fungi, and spores. In particular, HOCl is the active component responsible for pathogen disruption and inactivation by chlorine-releasing agents (CRAB). It is understood that the OCl− ion has little effect compared to undissolved HOCl, as the hypochlorite (OCl−), has only a minute effect compared to undissolved HOCl. Accordingly, the microbicidal effect of HOCl is the greatest when the percentage of undissolved HOCl is highest. In an aqueous solution of HOCl, ranging from approximately pH 4 to pH 7, chlorine exists predominantly as HOCl, whereas above pH 9, ClO− predominates.

Because HOCl is a highly active oxidizing agent, its mode of operation comprises destroying and/or deactivating cellular activity of proteins. For example, HOCl targets bacteria by chemically linking chlorine atoms to nucleotide bases that disrupt the function of bacterial DNA, impede metabolic pathways in which cells use enzymes to oxidize nutrients, and release energy, and other membrane-associated activities.

Additionally, HOCl has also been found to disrupt oxidative phosphorylation and other membrane-associated activity. Similarly, HOCl has been found to inhibit bacterial growth. For example, at 50 mM (2.6 ppm), HOCl completely inhibited the growth of E. coli within 5 minutes, including inhibiting the DNA synthesis by ninety-six percent. Unlike conventional antibiotics, the antimicrobial activity of HOCl is directly toxic to microbial cells, including many Gram-positive and Gram-negative bacteria and their biofilms. HOCl has demonstrated disinfection efficacy against eradication of bacteria, including Acinetobacter baumannii, Bacillus subtilis, Enterobacter cloacae, Enterococcus faecalis, Escherichia coli, Escherichia coli, Escherichia coli, Enterobacter, Klebsiella pneumoniae, Listeria monocytogenes, MRSA (Staph. aureus), Polymicrobial biofilm, Proteus vulgaris, Pseudomonas aeruginosa, Salmonella choleraesuis, Shigella flexneri, Staph epidermidis, and Yersinia enterocolitica.

Additionally, HOCl possesses viricidal activity properties. For example, it has been demonstrated that HOCl inactivated naked f2 RNA at the same rate as RNA in intact phage, whereas f2 capsid proteins could still adsorb to the host. HOCl has demonstrated disinfection efficacy against eradication of viruses including norovirus, filoviruses such as Ebola, and human coronaviruses like MERS-CoV and SARS, as well as fungi such as Candida and Aspergillus. Further, as a sporicide, HOCl causes the spore coat to detach from the cortex, where further degradation occurs.

Both topical and internal applications of HOCl are safe because it is the exact same substance white blood cells in the human body produce to fight infection. Indeed, extensive studies have demonstrated exceptional safety of HOCl. In particular, inhaling the aerosolized form of HOCl has also been shown to causes no adverse effects.

In some embodiments, the fluid preparation comprising at least in part HOCl may be administered intravenously as a treatment of sepsis, septicemia, and/or septic shock caused by microbial (including spores), viral, and fungal agents. For example, a solution of HOCl of low concentration levels and relatively low acidotic pH may be used as an intravenously administered disinfectant. In some embodiments, the fluid preparation comprising HOCl may include a concentration of approximately 0.01 to 0.05 percent of HOCl dissolved in a medium. In some embodiments, the aqueous solution of HOCl may include a concentration of approximately 0.01 percent of HOCl.

In some embodiments, the fluid preparation comprising at least in part HOCl may comprise a buffer solution to maintain the osmolarity of the pH level of the solution at a particular level. For example, a phosphate-buffered saline (PBS) may be included. In some embodiments, one part of 10×PBS may be diluted with nine parts of HOCL resulting in a 1× ph-adjusted solution of 1×PBS. In some embodiments, the resulting concentration of PBS may comprise approximately 137 mM of NaCl, approximately 2.7 mM of KCl, approximately 8 mM of Na2HPO4, and approximately 2 mM of KH2PO4. In some embodiments, prior to diluting HOCl with 10×PBS, the pH level of the one part of 10×PBS may be adjusted to approximately pH of 6.1 to 6.3.

In some embodiments, the buffer solution may include an acetate buffer, a citrate buffer, a borate buffer, and or other similar buffer. In some embodiments, the fluid preparation comprising at least in part HOCl and a buffer solution, as alluded to above, may have a pH level of 6.1 to 6.3.

In some embodiments, the fluid preparation comprising at least in part HOCl may be administered intravenously as an acute volume fluid manifested by hypovolemia associated with sepsis, septicemia, and/or septic shock. For example, a solution of HOCl of low concentration levels and relatively low acidotic pH may be used as a resuscitating fluid. In some embodiments, approximately 5 to 11 liters of the fluid preparation may be administered in the week after the manifestation of hypovolemia.

In some embodiments, the fluid preparation comprising at least in part HOCl may be administered intravenously as an acute volume fluid manifested by hypovolemia associated with one or more conditions. For example, the fluid preparation comprising at least in part HOCl may be administered intravenously as an acute volume fluid manifested by hemorrhagic shock, hypovolemic shock, burn injury, capillary leak syndrome, hypoalbuminemia, nephritic syndrome, and/or other conditions resulting in fluid loss.

In some embodiments, the fluid preparation comprising at least in part HOCl may be administered intravenously as a post-resuscitation fluid manifested by hypovolemia associated with one or more conditions, as alluded to above.

In some embodiments, the fluid preparation comprising at least in part HOCl may be administered intravenously as a post-resuscitation fluid manifested by hypovolemia associated with one or more conditions may include a therapeutically effective amount of hypochlorous acid within the aqueous solution comprises approximately 0.01% to 0.05% of the hypochlorous acid.

In some embodiments, the fluid preparation comprising at least in part HOCl may be combined with Human Serum Albumin of one or more concentrations. For example, the fluid preparation comprising HOCl may include a concentration of approximately 0.01 percent of HOCl dissolved in a medium (e.g., PBS) combined with Human Serum Albumin having a concentration level of approximately 5 to 10 percent by weight. In some embodiments, the fluid preparation comprising at least in part HOCl may be combined with Human Serum Albumin of one or more concentrations and with a solution comprising one or more amino acids having a concentration level of approximately 1 to 10 percent by weight.

In some embodiments, the fluid preparation comprising at least in part HOCl may be administered intravenously as an acute volume fluid manifested by sepsis-associated acute kidney injury (i.e., renal hypoperfusion). Often, renal hypoperfusion may be followed by renal failure especially in certain patient populations (i.e., those with existing renal dysfunctions or suffering from diabetes or other illnesses).

In some embodiments, the fluid preparation comprising at least in part HOCl may be used as a hemodialyzer agent during a renal dialysis in the event of renal failure, as alluded to above. For example, during a renal dialysis procedure, a dialysis machine may be configured to circulate blood from a patient through the dialysis machine to connect to a patient so that the blood from the patient may be cleansed through indirect exposure with a hemodialyzer agent. In some embodiments, the therapeutically effective supply of the hemodialyzer agent may be provided on a continuous basis.

In some embodiments, the fluid preparation comprising at least in part HOCl may be used as an agent during a peritoneal dialysis. For example, peritoneal dialysis may include a method for exchanging solutes and water in capillary vessels of a patient's peritoneal with hypertonic solution which is injected within the peritoneal cavity. Accordingly, during peritoneal dialysis, a diffusion of solutes which are transferred according to the concentration gradient and water migration due to the osmotic differences may occur.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method for treating acute hypovolemia due to one or more medical conditions, comprising:
injecting an aqueous solution comprising a therapeutically effective amount of hypochlorous acid to a patient in need thereof.

2. The method claim 1, wherein the therapeutically effective amount of hypochlorous acid within the aqueous solution comprises approximately 0.01% to 0.05% of the hypochlorous acid.

3. The method claim 1, wherein the one or more medical conditions comprise sepsis with shock, hemorrhagic shock, burn injury, nephritic syndrome, and multiorgan failure.

4. The method claim 3, wherein the one or more medical conditions are associated with third space fluid loss.

5. The method of claim 1, wherein the hypochlorous acid has a pH range of approximately 6.1 to 6.3.

6. The method of claim 1, wherein the hypochlorous acid is dissolved in a buffer solution.

7. The method of claim 6, wherein, the buffer solution comprises a phosphate buffered saline.

8. A solution for treating hypovolemia, comprising:
a first component comprising a therapeutically effective amount of a hypochlorous acid, wherein the hypochlorous acid has a pH of approximately 6.1 to 6.3.

9. The solution for treating hypovolemia of claim 8, wherein the solution is injected to a patient in need thereof.

10. The solution for treating hypovolemia of claim 8, further comprising:
a second component comprising a buffer with a buffering capacity for maintaining a pH of approximately 6.1 to 6.3.

11. The solution for treating hypovolemia of claim 10, wherein the buffer is selected from the group consisting of a phosphate buffer, acetate buffer, citrate buffer, borate buffer, and combinations thereof.

12. The solution for treating hypovolemia of claim 8, wherein the therapeutically effective amount of hypochlorous acid within the solution for treating hypovolemia comprises approximately 0.01% to 0.05% of the hypochlorous acid.

13. The solution for treating hypovolemia of claim 8, wherein the one or more medical conditions comprise sepsis with shock, hemorrhagic shock, burn injury, nephritic syndrome, and multiorgan failure.
